(19) United States
(12) Patent Application Publication
(10) Pub. No.: US 2013/0012443 A1
(43) Pub. Date: Jan. 10, 2013

(54) METHODS OF TREATMENT OF CENTRAL NERVOUS SYSTEM HEMORRHAGE USING PROTOPORPHYRIN IX-FE COMPOUNDS

(76) Inventor: Raymond F. Regan, Villanova, PA (US)

(21) Appl. No.: 13/542,046

(22) Filed: Jul. 5, 2012

Related U.S. Application Data
(60) Provisional application No. 61/505,216, filed on Jul. 7, 2011.

(51) Int. Cl. A61K 31/555; A61P 7/04; A61K 38/17; A61P 25/00; A61K 38/38; C07F 15/02

(52) U.S. Cl. 514/15.2; 514/185; 540/145; 514/15.3

(57) ABSTRACT

Described herein are methods and compositions relating to treatment of tissue damage resulting from a central nervous system (CNS) hemorrhage with a protoporphyrin IX-Fe compound.

### CROSS-REFERENCE TO RELATED APPLICATIONS

This application claims benefit under 35 U.S.C. §119(e) of U.S. Provisional Application No. 61/505,216 filed Jul. 7, 2011, the contents of which are incorporated herein by reference in their entirety.

### GOVERNMENT SUPPORT

The invention was made with Government support under Grant Number RO1 NS42273-08 awarded by the National Institutes of Health. The Government has certain rights in the invention.

### SEQUENCE LISTING

The instant application contains a Sequence Listing which has been submitted in ASCII format via EFS-Web and is hereby incorporated by reference in its entirety. Said ASCII copy, created on Jun. 29, 2012, is named 65973748.txt and is 25,159 bytes in size.

### TECHNICAL FIELD

The technology described herein relates to methods of treating hemorrhage in the central nervous system (CNS), such as in the brain of a subject. The technology described herein further relates to methods of treating hemorrhagic stroke in a subject.

### BACKGROUND

Hemorrhage can cause cell death in nervous tissue, which is at least partially mediated by the toxicity of hemoglobin breakdown products, including hemin. Furthermore, intracerebral hemorrhage can cause disruption of the blood-brain barrier, leading to tissue edema. Intracerebral hemorrhage accounts for 10-15% of the estimated 15 million worldwide incidences of stroke that occur yearly. Intracerebral hemorrhage has the highest morbidity and mortality of all stroke types and both intracerebral and subarachnoid hemorrhages have a higher mortality and higher lifetime cost than ischemic stroke.

The high mortality is due in part to the fact that CNS hemorrhage is a condition lacking productive treatment options. Surgical evacuation of the hematoma is not of benefit to most patients, presumably because the trauma of surgery negates the benefit of hematoma removal. To date, therapy is generally limited to supportive care including respiratory support, hydration, nutrition, and control of blood pressure. There is a distinct need for improved treatment options that can prevent the course of hemorrhagic damage in nervous tissue without exposing the subject to significant additional trauma.

### SUMMARY

Aspects of the technology described herein are directed to methods of treating a CNS hemorrhage in a subject, comprising administering to the subject a protoporphyrin IX-Fe compound.

Aspects of the technology described herein are based on the inventor's discovery that systemic administration of moderate quantities of a protoporphyrin IX-Fe compound confers a protective effect against the cellular injuries caused by CNS hemorrhage. Accordingly, there is provided herein a method of treating tissue damage resulting from a CNS hemorrhage or a complication thereof in a subject, the method comprising administering to the subject a protoporphyrin IX-Fe compound in a pharmaceutically acceptable carrier.

In some embodiments, the CNS hemorrhage is an intracranial hemorrhage. In some embodiments, the CNS hemorrhage is a cerebral or intracerebral hemorrhage. In some embodiments, the CNS hemorrhage is an intra-axial hemorrhage. In some embodiments, the CNS hemorrhage is an intraventricular hemorrhage. In some embodiments, the CNS hemorrhage is an intraparenchymal hemorrhage. In some embodiments, the CNS hemorrhage is an epidural hemorrhage. In some embodiments, the CNS hemorrhage is a subdural hemorrhage. In some embodiments, the CNS hemorrhage is a subarachnoid hemorrhage.

In some embodiments the complication resulting from a CNS hemorrhage is a stroke.

In some embodiments, the protoporphyrin IX-Fe compound is hemin. In some embodiments, the protoporphyrin IX-Fe compound is hematin. In some embodiments, the protoporphyrin IX-Fe compound is hemoglobin. In some embodiments, the protoporphyrin IX-Fe compound is methemoglobin. In some embodiments, the protoporphyrin IX-Fe compound is heme arginate. In some embodiments, the protoporphyrin IX-Fe compound is heme lysinate.

In some embodiments, the protoporphyrin DC-Fe compound is bound to albumin. In some embodiments, the protoporphyrin IX-Fe compound is bound to hemopexin. In some embodiments, the protoporphyrin IX-Fe compound is bound to haptoglobin.

In some embodiments, the protoporphyrin IX-Fe compound is conjugated or bound to a molecule which preferentially crosses the blood-brain barrier.

In some embodiments, the protoporphyrin DC-Fe compound is administered systemically. In some embodiments, the protoporphyrin IX-Fe compound is administered intravenously. In some embodiments, the protoporphyrin IX-Fe compound is administered intranasally.

In some embodiments, the protoporphyrin IX-Fe compound is administered locally to the site of the tissue damage.

In some embodiments, the method further comprises a step of diagnosing CNS hemorrhage in the subject prior to administering the protoporphyrin IX-Fe compound.

In some embodiments, the protoporphyrin IX-Fe compound is administered as one dose, 1-24 hours after the subject has experienced a CNS hemorrhage.

In some embodiments, the protoporphyrin IX-Fe compound is administered as one dose, 1-10 days after the subject has experienced a CNS hemorrhage.

In some embodiments, the protoporphyrin IX-Fe compound is administered as at least two doses with a time interval of 1-24 hours between the at least two doses.

In some embodiments, the protoporphyrin IX-Fe compound is administered as at least two doses with a time interval of 1-10 days between the at least two doses.

In some embodiments, the protoporphyrin IX-Fe compound is administered at a dose of 0.3 mg/kg to 100 mg/kg.

In some embodiments, the protoporphyrin IX-Fe compound is administered at a dose of 5 mg/kg to 75 mg/kg.

In some embodiments, the protoporphyrin IX-Fe compound is administered at a dose of 10 mg/kg to 50 mg/kg.

In some embodiments, the protoporphyrin IX-Fe compound is administered at a dose of 20 mg/kg to 30 mg/kg.

In some embodiments, the protoporphyrin IX-Fe compound is administered at a dose greater than 6 mg/kg/day.

Some aspects of the technology described herein comprise a pharmaceutical composition comprising a protoporphyrin IX-Fe compound for the treatment of CNS hemorrhage in a subject.

### DEFINITIONS

For convenience, the meaning of certain terms and phrases used in the specification, examples, and appended claims, are provided below. If there is an apparent discrepancy between the usage of a term in the art and its definition provided herein, the definition provided within the specification shall prevail.

The terms “decrease”, “reduced”, “reduction”, “inhibit” or “inhibition” are all used herein generally to mean a decrease by a statistically significant amount. However, for avoidance of doubt, “decrease”, “reduce”, “reduction”, “inhibition” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, or any decrease between 10-99% as compared to a reference level. In the context of a disease marker or symptom is meant a statistically significant decrease in such level. The decrease can be, for example, at least 10%, at least 20%, at least 30%, at least 40% or more, and is preferably down to a level accepted as within the range of normal for an individual without such disorder.

The terms “increased”, “increase” or “enhance” are all used herein to generally mean an increase by a statistically significant amount; for the avoidance of any doubt, the terms “increased”, “increase” or “enhance” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “administer” refers to the placement of a composition into a subject by a method or route which results in delivery of at least part of the administered composition to a desired site such that the desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, topical (including buccal and sublingual), intracranial, and intracerebral administration.

Exemplary modes of administration include, but are not limited to, injection, infusion, instillation, inhalation, or ingestion. “Injection” includes, without limitation, intravenous, intramuscular, intraarterial, intrathecal, intraventricular, intraorbital, intracardiac, intradermal, intraperitoneal, subcutaneous, subarachnoid, intraspinal, intracerebrospinal, intracranial, intracerebral, and infusion injection.

As used herein in the context of expression, the terms “treat,” “treatment,” and the like, as used in the context of the therapeutic methods described herein, refer to a decrease in severity, indicators, symptoms, and/or markers of CNS hemorrhage as described herein. In the context of the present technology insofar as it relates to any of the conditions recited herein, the terms “treat,” “treatment,” and the like mean to relieve, alleviate, ameliorate, inhibit, slow down, reverse, or stop the progression, aggravation, deterioration, anticipated progression or severity of at least one symptom or complication associated with CNS hemorrhage. In one embodiment, the symptoms of CNS hemorrhage are alleviated by at least 10%, at least 20%, at least 30%, at least 40%, or at least 50%.

As used herein, the phrase “therapeutically effective amount”, “effective amount” or “effective dose” refers to an amount that provides a therapeutic benefit in the treatment or management of CNS hemorrhage, e.g. an amount that provides a statistically significant decrease in at least one symptom, indicator and/or marker of CNS hemorrhage. Determination of a therapeutically effective amount is well within the capability of those of ordinary skill in the art. Generally, a therapeutically effective amount can vary with the subject's history, age, condition, and gender, as well as the severity and type of the medical condition in the subject, and administration of other pharmaceutically active agents.

As used herein, the term “pharmaceutical composition” refers to the active agent in combination with a pharmaceutically acceptable carrier as commonly used in the pharmaceutical industry.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient or is toxic to the subject, use thereof in the therapeutic compositions is contemplated. Supplementary active ingredients can also be incorporated into the compositions.

As used herein, the term “hemorrhage” refers to the escape of blood from the intravascular space.

As used herein, the term “nervous tissue” refers to tissue (comprising nerve fibers, neurons, neuron support cells, Schwann cells, dendrites, glial cells, astrocytes, oligodendrocytes and supporting tissues) that initiate and transmit nerve impulses. The term includes nervous tissue present in both the central nervous system and the peripheral nervous system, and comprises any or all of the following: axons, dendrites, fibrils, ganglion cells, granule cells, grey matter, myelin, neuroglial cells, neurolemma, neuronal cells or neurons, Schwann cells, stellate cells, and white matter.

As used herein, a “neuron” is a conducting or nerve cell of the nervous system that typically consists of a cell body (perikaryon) that contains the nucleus and surrounding cytoplasm; several short, radiating processes (dendrites); and one long process (the axon), which terminates in twig-like branches (telodendrons), and which may have branches (collaterals) projecting along its course. Examples of neurons include, without limitation, autonomic neurons, neurons of the dorsal root ganglia (DRG), enteric neurons, interneurons, motor neurons, peripheral neurons, sensory neurons, and neurons of the spinal cord.

As used herein, the “central nervous system” refers to the nervous tissue of a subject that comprises the brain and the spinal cord. The central nervous system can also include the retina and the cranial nerves.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above. In certain embodiments, the subject is a mammal, e.g., a primate, e.g., a human.

Preferably, the subject is a mammal. The mammal can be a human, non-human primate, mouse, rat, dog, cat, horse, or cow, but is not limited to these examples. Mammals other than humans can be advantageously used as subjects that represent animal models of CNS hemorrhage. In addition, the methods described herein can be used to treat domesticated animals and/or pets. A subject can be male or female. A subject can be one who has been previously diagnosed with or identified as suffering from or having CNS hemorrhage or one or more complications related to CNS hemorrhage, and optionally, but need not have already undergone treatment for CNS hemorrhage or the one or more complications related to CNS hemorrhage. A subject can also be one who has been diagnosed with or identified as suffering from CNS hemorrhage or one or more complications related to CNS hemorrhage, but who shows improvements in known CNS hemorrhage risk factors as a result of receiving one or more treatments for CNS hemorrhage or for one or more complications related to CNS hemorrhage. Alternatively, a subject can also be one who has not been previously diagnosed as having CNS hemorrhage or one or more complications related to CNS hemorrhage. For example, a subject can be one who exhibits one or more risk factors for CNS hemorrhage or one or more complications related to CNS hemorrhage or a subject who does not exhibit CNS hemorrhage risk factors.

The term “statistically significant” or “significantly” refers to statistical significance and generally means a difference of at least two standard deviations (2SD).

Other than in the operating examples, or where otherwise indicated, all numbers expressing quantities of ingredients or reaction conditions used herein should be understood as modified in all instances by the term “about.” The term “about” when used in connection with percentages can mean±1%.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIGS. 1A and 1B depict graphs demonstrating that systemic hemin therapy decreases blood-brain barrier disruption when administered after experimental intracerebral hemorrhage (ICH). FIG. 1A depicts a graph demonstrating that ICH was induced by striatal autologous blood injection (n=7-9/condition). Mice were treated with 26 mg/kg hemin i.p. 1 hour later, as a one-time dose, or repeated in 24 hours; controls received vehicle i.p. only. Evan's blue leakage into the striatal parenchyma, a marker of blood-brain barrier disruption, was quantified at 3 days. FIG. 1B depicts a graph demonstrating that ICH was induced by collagenase injection (n=3-5/condition); mice received 26 mg/kg hemin 1 or 3 hours after collagenase, and again 24 h later, or an equal volume of vehicle. Sham mice received anesthesia and needle trauma only. *P<0.05, **P<0.01, hemin treated v. vehicle-treated controls.

FIG. 2 depicts a graph demonstrating that systemic hemin therapy increases peri-hematomal cell viability after striatal hemorrhage. Intracerebral hemorrhage was modeled in 2-3 month old mice (5-6/condition) by striatal injection of autologous blood (25 μl) or collagenase (0.014 units), followed 1 or 3 hours later by 4 mg/kg hemin i.p. (repeated 24 h later) or vehicle. Five days after ICH, striatal cell viability was quantified by MTT assay. *P<0.05, **P<0.01, hemin treated v. vehicle-treated controls.

FIG. 3 depicts experimental results indicating that hemin treatment increases expression of the protective enzyme heme oxygenase-1 (HO-1) in the mouse striatum. Mice received 26 mg/kg hemin i.p for 1 or 2 days, or an equal volume of saline. Striatal HO-1 expression was assessed 24 hours after the last hemin dose. Actin levels provide a gel-loading control. *P<0.05 v. expression in saline-treated controls, n=4/conditon. The graph shows the quantification of the gel band densities.

FIG. 4 depicts experimental results indicating that hemin or heme arginate injection increases HO-1 expression in the mouse striatum (STR) or cortex (CTX). Mice received 26 mg/kg hemin or heme arginate i.p for 1 or 2 days, or an equal volume of saline. HO-1 expression was assessed 24 hours after last dose. Actin serves as a control.

### DETAILED DESCRIPTION

The technology described herein is, at least in part, based on the discovery that protoporphyrin IX-Fe compounds, when administered to a subject having suffered CNS tissue damage resulting from hemorrhage, alleviate the symptoms of the hemorrhage or complications resulting from it in an established mouse model for intracerebral hemorrhage. Specifically, it is demonstrated herein that protoporphyrin IX-Fe compounds increase expression of the protective enzyme heme oxygenase-1 (HO-1) in the mouse striatum and that hemin treatment surprisingly increased striatal cell viability after experimental intracerebral hemorrhage.

Accordingly, aspects of the technology described herein can be directed to methods of treating a CNS hemorrhage in a subject, comprising administering to the subject a protoporphyrin IX-Fe compound. In some embodiments the administration is intravenous. In some embodiments the administration is intranasal.

The technology described herein is particularly surprising in view of the report by Wang and Doré (Brain. 2007 June; 130(Pt 6):1643-52) who described that heme oxygenase-1 (HO-1) induction is detrimental after intracerebral hemorrhage. Wang and Doré observed that HO-1 was induced in cells surrounding a hematoma in wild-type mice. These mice sustained more perihematomal injury and neurological deficits than HO-1 knockout mice. Although Wang and Doré did not test the effect of hemin treatment, induction of HO-1 in our model by hemin has an effect that is opposite of that expected based on the teaching of Wang and Doré.

One source of damage caused by CNS hemorrhage is believed to be the toxicity of hemoglobin breakdown products. In particular, heme (protoporphyrin IX-Fe²⁺) and hemin (the chloride salt of protoporphyrin IX-Fe³⁺) have been shown to be toxic to cells at concentrations 500-1000 times lower than those found at the site of a CNS hemorrhage. Hemin is a pro-oxidant and toxicity results from the release of redox-active iron, the depletion of cellular stores of NADPH and glutathione, production of superoxide and hydroxyl radicals, and the peroxidation of membrane lipids (Robinson et al., Redox Report 2009 14:228-235). In nervous tissue, cell death is observed and the blood-brain barrier can be disrupted.

Heme oxygenase-1 (HO-1) is known to degrade hemin to biliverdin, iron, and carbon monoxide. Early work in the field found that upregulation of HO-1 protected cultured astrocytes against toxic levels of hemin (Regan et al. Neuroscience 2002, 113:985-994) and that low doses of hemin could induce HO-1 expression in cultured cells (Da Silva et al. J Lab Clin Med 1996, 128:290-296). Subsequent studies found, however, that cultured neurons displayed the opposite response to HO-1 activity in the presence of toxic levels of hemin, resulting in greater neuronal cell death when HO-1 is active in the presence of hemin. (Benvenisti-Zarom et al. Neuroscience Letters 2006, 398:230-4; Robinson et al., Redox Report 2009 14:228-235).

Certain aspects of the technology described herein relate to methods of treating CNS hemorrhage in a subject by administering to the subject a protoporphyrin IX-Fe compound. As used herein the term “protoporphyrin IX-Fe compound” refers to a compound comprising protoporphyrin IX (Formula I) and an iron ion. The iron ion present in a protoporphyrin DC-Fe compound can be either Fe²⁺ or Fe³⁺. In some embodiments, a protoporphyrin IX-Fe compound can be heme (protoporphyrin IX-Fe²⁺). In some embodiments, a protoporphyrin DC-Fe compound can be hemin (chloride salt of protoporphyrin IX-Fe³⁺; Formula II). In some embodiments, a protoporphyrin IX-Fe compound can be hematin (protoporphyrin IX-Fe³⁺ hydroxide).

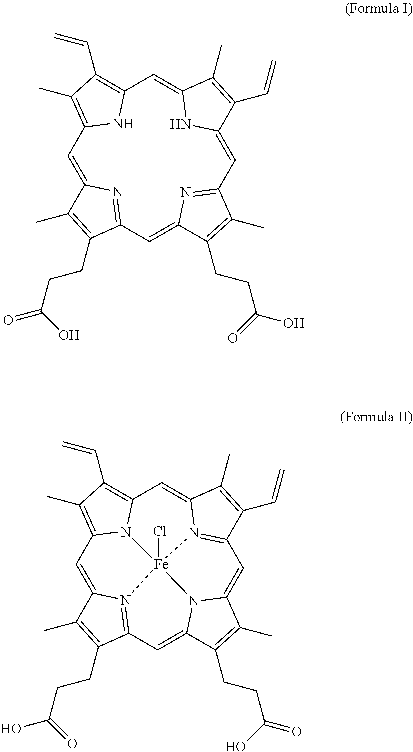

In some embodiments, the protoporphyrin DC-Fe compounds useful in the methods of the technology described herein include, but are not limited to, hemin (Formula II), hematin, heme arginate, heme lysinate, hemoglobin and methemoglobin. In some embodiments, a protoporphyrin IX-Fe compound can be a breakdown product of hemoglobin and/or methemoglobin. In some embodiments, a protoporphyrin IX-Fe compound can be a metabolized form of hemoglobin and/or methemoglobin.

In some embodiments, a protoporphyrin IX-Fe compound can additionally comprise a further ion and/or conjugate.

In some embodiments, a protoporphyrin IX-Fe compound can be bound or conjugated to albumin prior to administration. In some embodiments, a protoporphyrin IX-Fe compound can be bound or conjugated to hemopexin prior to administration. In some embodiments, a protoporphyrin IX-Fe compound can be bound or conjugated to haptoglobin prior to administration.

Protoporphyrin IX-Fe compounds useful in the methods described herein include bioprecursors or compounds which may be converted in an animal body into protoporphyrin IX-Fe compounds. Protoporphyrin IX-Fe compounds can be in the form of pharmaceutically acceptable salts, esters, hydrates, and solvates of a protoporphyrin IX-Fe compound as described herein.

Variations and modifications to a protoporphyrin IX-Fe compound can provide means for targeting. For example, a protoporphyrin IX-Fe compound can be linked with a molecular counter-ligand, including, for example, molecules which target the nervous tissue, to permit the protoporphyrin IX-Fe compound to accumulate preferentially or specifically in that tissue. In some embodiments, a protoporphyrin IX-Fe compound can be bound to a molecule which preferentially crosses the blood-brain barrier.

Protoporphyrin IX-Fe compounds can be synthesized by methods familiar to those skilled in the art, such as described in Tenhunen et al, J. Pharm Pharmacol. 39:780-86 which is incorporated herein by reference in its entirety. Protoporphyrin IX-Fe compounds can also be purchased commercially, e.g. PANHEMATIN® (Lundbeck Inc; Deerfield, Ill.), hematin (Catalog No. H3281, Sigma-Aldrich; St. Louis, Mo.) and hemin (Catalog No. H9039 and 51280, Sigma-Aldrich; St. Louis, Mo.).

Protoporphyrin IX-Fe compounds include derivatives of protoporphyrin IX-Fe compounds as described herein. Protoporphyrin IX-Fe compounds can include, but are not limited to, perfluoromethyl side chain substituted derivatives, 2,4 substituted heme substitutents, meso-substituted heme substituents, deuteron-substituted heme substitutents, diacetyldeutero-substituted heme substituents, hydroxyl coordinated hemin, 1,2-dimethyl imidazole coordinated hemin, synthetic polymer-bound hemin derivatives, hemin thienyl ester, deuterohemin, ferri-heme undecapeptide, hemin-sepharose, etiohemin, ferriprotoporphyrin IX-chloroquine complex, hemin-CN, 2,4-dimethyldeuterohemin, octaethylporphyrinatoiron(III)perchlorate, octaethylporphyrinato-iron(III)perchlorate, monoimidazole adduct, pemptohemin, isopemptohemin, hemin dimethyl ester, alpha-oxyprotohemin IX, hematohemin IX, bis(glutathione dimethyl ester)-hemin complex, chloroprotohemin IX, 1,4,5,8-tetramethylhemin, nitro-etioheme-1, hemin dicyanide, and n-butyletiohemin I. Methods of producing such compounds are known in the art (Shibata et al., JACS 2010, 132:6091-8; Singh et al., BBA 1998 1384:103-111; Boffi et al., Biophysical Journal 1999 77:1143-9; Uotani et al., J Inorg Biochem 1984 22:85-9; Ohtaki et al., Solid State Ionics 1996, 86-88:333-336; Biochem J 1978, 174:893; Europ J Biochem 1976, 71:613; Anal Biochem 1982, 121:244; Biochim Biophys Acta 1989, 996:226; Biochim Biophys Acta 1991, 1074:19; Arch Biochim Biophys 1993, 306: 158-62; J Biol Chem 2002 277:33018-31; Biochim Biophys Acta 1980, 621:19; J Biol Chem 1981, 256:6075; Biochim Biophys Acta 1981, 637:231; PNAS 1986, 83:531; Eur J Biochem 1986, 156:179; Biochem Biophys Res Commun 1990, 169:22; Biochem Biophys Res Commun 1991, 178:95; Biochim Biophys Acta 1992, 1117:243-250; J Am Chem Soc 2001, 123:8080-8; FEBS Lett 2005, 579:271-274; J Biol Inorg Chem 2005, 10:283-293). Derivatives of protoporphyrin IX-Fe compounds useful in the methods described herein will retain the ability to increase neuronal cell viability following a CNS hemorrhage. In addition or in the alternative, the compound administered can increase HO-1 activity or expression as measured by the methods described elsewhere herein.

In some embodiments, a protoporphyrin IX-Fe compound can be bound to albumin. Hemin readily binds to albumin. In some embodiments, the protoporphyrin IX-Fe compound bound to albumin is hemin. When bound to albumin, hemin causes less blood vessel inflammation and damage (phlebitis) and is more stable (Anderson et al. Ann Intern Med 2005 142:439-450).

Albumin can be mammalian in origin. In some embodiments, the albumin is human. In some embodiments, the albumin is bovine. By way of non-limiting example, the albumin can be from goat, baboon, chicken, guinea pig, mouse, rabbit, or rat. In some embodiments the albumin can be produced in a transgenically-modified organism. Preferably, the albumin is from the same species as the subject which is to be treated according to the methods described herein.

The sequences of the albumin protein (e.g. the human albumin amino acid sequence, NCBI Accession No: NP_{—}000468 (SEQ ID NO: 1)) and the gene encoding the albumin protein (e.g. the human albumin mRNA, NCBI Accession No: NM_{—}000477 (SEQ ID NO:2)) are known to those skilled in the art. Alternatively, albumin is available commercially, e.g. bovine albumin (Catalog No: A7030) and human albumin (Catalog No: A3782) are available from Sigma-Aldrich (St. Louis, Mo.).

In some embodiments, a protoporphyrin IX-Fe compound can be bound to haptoglobin. In some embodiments, the protoporphyrin IX-Fe compound bound to haptoglobin is hemoglobin. In some embodiments, the protoporphyrin IX-Fe compound bound to haptoglobin is methemoglobin.

Haptoglobin is a protein that actively binds both hemoglobin and methemoglobin. The resulting complex is then taken up by a number of cell types.

Haptoglobin useful in the methods described herein can be mammalian in origin. In some embodiments, the haptoglobin is human. In some embodiments, the haptoglobin is murine. By way of non-limiting example, the haptoglobin can be from a rabbit or a rat. In some embodiments the haptoglobin can be produced in a transgenically-modified organism. Preferably, the haptoglobin is from the same species as the subject which is to be treated according to the methods described herein.

The sequences of the haptoglobin protein (e.g. the human haptoglobin amino acid sequence, NCBI Accession Nos: NP_{—}005134 (SEQ ID NO:3) and NP_{—}001119574 (SEQ ID NO:4)) and the gene encoding the haptoglobin protein (e.g. the human haptoglobin mRNA, NCBI Accession Nos: NM_{—}005143 (SEQ ID NO:5) and NM_{—}001126102 (SEQ ID NO:6)) are known to those skilled in the art. Alternatively, haptoglobin is available commercially, e.g. human haptoglobin (Catalog No: H3536 is available from Sigma-Aldrich (St. Louis, Mo.).

In some embodiments, a protoporphyrin IX-Fe compound can be bound to hemopexin. In some embodiments, the protoporphyrin IX-Fe compound bound to hemopexin is hemin.

Hemopexin is the protein with the highest known affinity for hemin and is present in plasma and other bodily fluids. Hemopexin is a protective protein that prevents accumulation of oxidative species and the depletion of iron in the body. The hemin-hemopexin complex is taken up by cells that express the LRP1 (LDL receptor-related protein 1) receptor. LRP1 is expressed in a number of cell types, notably including neurons. LRP1 has been implicated in transport of some molecules across the blood-brain barrier (Hong et al., Neuropharmacology 2009 56:1054-9).

Administration of hemin, particularly recurrent administration of hemin, to a subject can deplete the endogenous level of hemopexin. Furthermore, some disease processes correlated with a high rate of hemorrhagic stroke, in particular sickle cell disease, are characterized by low hemopexin levels.

Thus, administration of a protoporphyrin IX-Fe compound bound to hemopexin can increase uptake of the protoporphyrin IX-Fe compound by certain cell types and is proposed to avoid deleterious depletion of endogenous hemopexin.

Hemopexin useful in the methods described herein can be mammalian in origin. In some embodiments, the hemopexin is human. In some embodiments, the hemopexin is murine. By way of non-limiting example, the hemopexin can be from a rabbit or a rat. In some embodiments the hemopexin can be produced in a transgenically-modified organism. Preferably, the hemopexin is from the same species as the subject which is to be treated according to the methods described herein.

The sequences of the hemopexin protein (e.g. the human hemopexin amino acid sequence, NCBI Accession No: NP_{—}000604 (SEQ ID NO:7)) and the gene encoding the hemopexin protein (e.g. the human hemopexin mRNA, NCBI Accession No: NM_{—}000613 (SEQ ID NO:8)) are known to those skilled in the art. Alternatively, hemopexin is available commercially, e.g. human hemopexin (Catalog No: H9291 is available from Sigma-Aldrich (St. Louis, Mo.).

The dosage of a protoporphyrin IX-Fe compound can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen.

The dosage ranges for the administration of a protoporphyrin IX-Fe compound depend upon the form of the protoporphyrin IX-Fe compound, and its potency, as described further herein, and are amounts large enough to produce the desired effect in which the symptoms, markers, or signs of CNS hemorrhage are reduced. The dosage should not be so large as to cause substantial adverse side effects. Generally, the dosage can vary with the age, condition, and sex of the patient and can be determined by one of ordinary skill in the art. The dosage can also be adjusted by the individual physician in the event of any complication or based upon the subject's sensitivity to the protoporphyrin IX-Fe compound. Typically, the dosage ranges from 0.001 mg/kg body weight to 100 mg/kg body weight. In some embodiments, the dose range is from 0.3 mg/kg body weight to 100 mg/kg body weight. In some embodiments, the dose range is from 5 mg/kg body weight to 75 mg/kg body weight. In some embodiments, the dose range is from 10 mg/kg body weight to 50 mg/kg body weight. In some embodiments, the dose range is from 20 mg/kg body weight to 30 mg/kg body weight. In some embodiments, the dose range is greater than 6 mg/kg body weight/day.

A composition comprising a protoporphyrin IX-Fe compound can be administered over a period of time, such as over a 5 minute, 10 minute, 15 minute, 20 minute, or 25 minute period. The administration can be repeated, for example, on a regular basis, such as hourly for 3 hours, 6 hours, 12 hours or longer or such as biweekly (i.e., every two weeks) for one month, two months, three months, four months or longer. When multiple doses are administered, the doses can be separated from one another by, for example, one hour, three hours, six hours, eight hours, one day, two days, one week, two weeks, or one month.

After an initial treatment regimen, the treatments can be administered on a less frequent basis. For example, after administration biweekly for three months, administration can be repeated once per month, for six months or a year or longer. In some embodiments, administration can be chronic, e.g., one or more doses daily over a period of weeks or months.

Administration of a composition comprising a protoporphyrin IX-Fe compound can reduce levels of a marker or symptom of CNS hemorrhage, e.g. headache, seizures or motor, sensory or cognitive impairment by at least 10%, at least 15%, at least 20%, at least 25%, at least 30%, at least 40%, at least 50%, at least 60%, at least 70%, at least 80% or at least 90% or more.

It is to be understood that, for any particular subject, specific dosage regimes should be adjusted over time according to the individual need and the professional judgment of the person administering or supervising the administration of the compositions. For example, the dosage of the therapeutic can be increased if the lower dose does not provide sufficient therapeutic activity. Effective doses may be extrapolated from dose-response curves derived from, for example, animal model test bioassays or systems.

Dosages for a particular patient or subject can be determined by one of ordinary skill in the art using conventional considerations (e.g. by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum or tissue half-life of the protoporphyrin IX-Fe compound as disclosed herein, or functional derivatives thereof, and the condition of the patient, as well as, for example, the body weight of the patient to be treated. The size of the dose is also determined by the existence, nature, and extent of any adverse side-effects that accompany the administration of a particular composition, formulation, or the like in a particular subject. Therapeutic compositions comprising a protoporphyrin IX-Fe compound or functional derivatives thereof are optionally tested in one or more appropriate in vitro and/or in vivo animal models of disease, such as the mouse model of intracerebral hemorrhage described herein, to confirm efficacy, evaluate tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD₅₀ of the relevant formulation, and/or observation of any side-effects of a protoporphyrin IX-Fe compound or functional derivatives thereof at various concentrations, e.g., as applied to the mass and overall health of the patient. In determining the effective amount of a protoporphyrin IX-Fe compound or functional derivatives thereof to be administered in the treatment of CNS hemorrhage, the physician evaluates, among other criteria, circulating plasma levels, formulation toxicities, and progression of the condition.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred. A dose may be formulated in animal models to achieve a circulating plasma concentration range that includes the IC₅₀ (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage of such compounds lies preferably within a range of circulating concentrations that include the ED₅₀ with little or no toxicity. The dosage may vary within this range depending upon the dosage form employed and the route of administration utilized.

With respect to the therapeutic methods of the technology described herein, unless otherwise specified, it is not intended that the administration of the protoporphyrin IX-Fe compound be limited to a particular mode of administration, dosage, or frequency of dosing; the embodiments described herein contemplate all modes of administration, including intramuscular, intravenous, inhalation, intranasal, intraperitoneal, intravesicular, intraarticular, intralesional, subcutaneous, or any other route sufficient to provide a dose adequate to treat the CNS hemorrhage.

A number of protoporphyrin IX-Fe compounds are readily available. For example, PANHEMATIN® containing a dose of 1 to 4 mg/kg/day of hematin can be given over a period of 10 to 15 minutes for 3 to 14 days based on the clinical signs of CNS hemorrhage.

Certain aspects of the technology described herein relate to administering a protoporphyrin IX-Fe compound to a patient having a CNS hemorrhage. The CNS hemorrhage can be, e.g., an intracranial hemorrhage, a cerebral hemorrhage, an intracerebral hemorrhage, an intra-axial hemorrhage, an intraventricular hemorrhage, an intraparenchymal hemorrhage, an epidural hemorrhage, a subdural hemorrhage and/or a subarachnoid hemorrhage.

In some embodiments, the technology described herein comprises first diagnosing the subject, such as a human patient, as having suffered from a CNS hemorrhage prior to administering the protoporphyrin IX-Fe compound to the subject.

Subjects having a CNS hemorrhage can be identified by a physician using current methods of diagnosing CNS hemorrhage. Symptoms and/or complications of a CNS hemorrhage which characterize these conditions and aid in diagnosis include, but are not limited to, seizures, paralysis, sudden changes in vision, abnormal sense of taste, unconsciousness, lethargy, apathy, difficulty speaking, difficulty swallowing, headache, loss of coordination, loss of balance, tremors, loss of fine motor skills, weakness, difficulty reading or writing, nausea and swelling of the optic nerve. Tests that may aid in a diagnosis of CNS hemorrhage include, but are not limited to, CT scan, CT scan angiography, cerebral angiography, complete blood count, MRI, platelet counts, prothrombin time (PT) test, and partial thromboplastin time (PTT) test.

CNS hemorrhage is a common problem in premature births and ultrasound is particularly useful in diagnosing CNS hemorrhage in these neonate subjects.

In some embodiments, the diagnosis of CNS hemorrhage applied to the methods described herein comprises a CT scan or CT scan with angiography.

Subjects at risk of developing or having a CNS hemorrhage include subjects having or diagnosed as having or at risk of having trauma, stroke, high blood pressure, infection, a tumor, a blood clotting deficiency, and blood vessel abnormalities. CNS hemorrhage can also be idiopathic.

The individual with an intracranial hemorrhage is often unconscious or dazed or otherwise unable to give a complete medical history. The physician may need to rely on those who were with the individual when the event occurred, as well as friends or family members, to provide information about the individual's current and past medical conditions and diseases. In this case, the history may be inaccurate or incomplete for past injuries, illnesses, surgical procedures, and current treatment of existing chronic diseases.

Many individuals with an epidural hemorrhage caused by an arterial tear become unconscious at the trauma scene and then experience a brief period of consciousness referred to as a lucid interval. This is followed by a decrease in the level of consciousness. Other individuals never regain consciousness, and others are awake but dazed. Symptoms include headache, vomiting, and seizures.

Typically, individuals with a subdural hemorrhage report having a headache. Drowsiness, confusion, and a decreasing level of consciousness are evident. The individual may remember experiencing a bump on the head or some other head trauma in the recent past, but frequently no obvious traumatic injury has occurred.

Symptoms of subarachnoid hemorrhage may include a sudden onset of severe headache, nausea, vomiting, stiff neck (nuchal rigidity), fainting, and sensitivity to light (photophobia). Occasionally, an individual may experience warning symptoms that indicate a cerebral aneurysm is leaking or about to rupture, including headache (sentinel headache), weakness on one side of the body, numbness, tingling, speech disturbance, and double vision that does not go away. Some individuals with a ruptured cerebral aneurysm may complain of a severe headache and fall unconscious almost immediately. Others may experience a headache but remain conscious. Still others may suddenly become unconscious without a headache and without warning.

Individuals with intracerebral hemorrhage may have a history of hypertension, diabetes, or treatment with anticoagulants. Symptoms of intracerebral hemorrhage typically come on during the day and include progressive deterioration in consciousness (50% of cases), nausea and vomiting (40% to 50% of cases), headache (40% of cases), seizures (6% to 7% of cases), weakness or paralysis on one side (including face, arm, and leg), slurred speech, difficulty expressing themselves in words (expressive aphasia) or understanding speech (receptive aphasia), disturbances in eye movement, difficulty swallowing (dysphagia), or respiratory depression.

The physician or other examiner or the patient may observe changes in the individual's mental status and level of consciousness that may range from clouding of consciousness, confusion, lethargy, obtundation, and stupor to coma. Strength testing may reveal weakness or paralysis on one side. The individual may vomit and have seizures. Speech may be disturbed. Elevated pressure inside the cranium (intracranial pressure [ICP]), and thus in the brain and CSF, may result in pupils that appear unequal in size and react sluggishly to light.

Computed tomography (CT) is the standard diagnostic tool to quickly determine the presence of skull fractures and bleeding within the skull. If the CT is negative for bleeding, lumbar puncture is performed to determine if blood is present in the CSF. Magnetic resonance imaging (MRI) is not used in the acute phase of injury but is useful after the initial 48 hours to assess the extent of injury to the brain. If a ruptured aneurysm is suspected, a complete vascular study (arteriography) of the carotid and cerebral arteries helps pinpoint the location of the ruptured aneurysm. An angiography may also be performed if subarachnoid hemorrhage is suspected. Additional diagnostic tests that may be relevant to establishing a diagnosis and treatment plan for CNS hemorrhage may include an electrocardiogram (ECG), x-ray, urinalysis, and blood studies (complete blood count [CBC], prothrombin time [PT], erythrocyte sedimentation rate [ESR], blood glucose, electrolytes, and blood type). A diagnosis of subdural hemorrhage/hematoma may require additional tests because symptoms are similar to those of many other diseases and conditions.

In some embodiments, the pharmaceutical composition comprising a protoporphyrin IX-Fe compound comprises additional agents to treat a CNS hemorrhage.

Efficacy of treatment can be assessed, for example by measuring a marker, indicator, or symptom of CNS hemorrhage as described herein or any other measurable parameter appropriate. It is well within the ability of one skilled in the art to monitor efficacy of treatment or prevention by measuring any one of such parameters, or any combination of parameters.

Effective treatment is evident when there is a statistically significant improvement in one or more markers, indicators, or symptoms of CNS hemorrhage, or by a failure to worsen or to develop symptoms where they would otherwise be anticipated. As an example, a favorable change of at least 10% in a measurable parameter of CNS hemorrhage, and preferably at least 20%, 30%, 40%, 50% or more can be indicative of effective treatment. Efficacy for a given protoporphyrin IX-Fe compound or formulation of that drug can also be judged using an experimental animal model known in the art for a condition described herein. When using an experimental animal model, efficacy of treatment is evidenced when a statistically significant increase in a marker is observed, e.g. the extent of cell death following an induced CNS hemorrhage.

In some embodiments, the protoporphyrin LX-Fe compounds are administered systemically. In some embodiments, the administration of the protoporphyrin IX-Fe compounds is intravenous. Typically, when systemic administration is used, the diagnosis of CNS hemorrhage is first confirmed.

Systemic administration includes, for example, extracranial, intravenous, intramuscular, intraperitoneal or parenteral administration.

In some embodiments, the administration is intracranial, intracerebral or other to selected area of the brain, or in general to brain extracellular fluid. The selection of the area can in some aspects be determined by diagnosing the location of the CNS hemorrhage prior to administering the protoporphyrin IX-Fe compound to the subject. If a location of the CNS hemorrhage is diagnosed, the administration can be directly to the location of the CNS hemorrhage. This can be accomplished using targeted methods well known to one skilled in the art, and include, for example, stereotactical injections of the protoporphyrin IX-Fe compound.

In some embodiments, the administration can be into intracranial vessels.

In some embodiments, the administration is intranasal. Intranasal administration can bypass the blood-brain barrier (Dhuria et al J Pharm Sci 2010 99:1654-73).

The protoporphyrin IX-Fe compounds can be administered as a one time injection or as two or more injections having a time period in between. For example, the protoporphyrin IX-Fe compound can be administered to the subject as soon as the diagnosis of CNS hemorrhage is made. The protoporphyrin IX-Fe compound can be administered about 1, 2, 3, 4, 5, 6, 7, 8, 9, or days after the CNS hemorrhage has occurred. In some embodiments, the protoporphyrin IX-Fe compound can be administered 30 minutes to 24 hours, such as at about hour 1, 2, 3, 4, 5, 6, 7, 8, 9, 10, 11, 12, 13, 14, 15, 16, 17, 18, 19, 20, 21, 22, 23, or 24 after the CNS hemorrhage occurred or after it was diagnosed or confirmed.

The protoporphyrin IX-Fe compounds can also be administered in time intervals. For example, one can administer 2, 3, 4, 5, 6, 7, 8, 9, or even 10 injections about 1, 2, 3, 4, 5, 6, 7, 8, 9, 10, 11, 12, or 12-24 hours apart. One can also use intervals between 1-10 days, such as 1, 2, 3, 4, 5, 6, 7, 8, 9, or 10 days.

In some embodiments, the injections are repeated until the symptoms of the CNS hemorrhage decrease.

In some embodiments, the injections are repeated until the symptoms of the CNS hemorrhage are significantly reduced so that the subject can function independently if physical or mental function was affected by the CNS hemorrhage.

In some embodiments, the injections are repeated until the subject has returned to a normal or near normal condition as exhibited prior to the occurrence of the CNS hemorrhage. Subjective and objective criteria can be used in determining the “normal condition” for each individual.

The blood-brain barrier (BBB) refers to the structures separating the bloodstream and cerebrosprinal fluid (CSF) in the CNS of a subject. The capillaries within in the CNS are surrounded by thick basement membranes, astrocytic endfeet, endothelial cells, and tight cell junctions not seen along capillaries elsewhere in the body. The BBB inhibits the entry of foreign bodies (e.g. bacterial cells) or certain molecules (e.g. large or hydrophilic molecules) into the CSF while allowing the diffusion of small molecules (e.g. oxygen, carbon dioxide, hormones). Active transport of proteins or molecules such as glucose also occurs across the BBB.

It has been recognized that BBB disruption is a hallmark of intracerebral hemorrhage-induced brain injury. Such disruption contributes to edema formation, the influx of leukocytes, and the entry of potentially neuroactive agents into the perihematomal brain, all of which may contribute to brain injury (see, e.g., Keep et al. Cerebral Hemorrhage: Acta Neurochirurgica Supplementum, 2008, Volume 105, Part 3, 73-77). When there is a breakdown of the BBB, even locally due to CNS hemorrhage, systemic administration of the compounds of the technology described herein can result in delivery of the compounds through the BBB, likely at the site of tissue damage.

However, in some embodiments, a protoporphyrin IX-Fe compound can be bound to a molecule which preferentially crosses the blood-brain barrier in order to facilitate the compound's access to brain tissue. Such molecules include, but are not limited to peptidomimetic monoclonal antibodies that bind the tranferrin receptor and ascorbic acid. In some embodiments, a protoporphyrin IX-Fe compound is administered to a subject such that a substantial portion of the protoporphyrin IX-Fe compound crosses the blood-brain barrier. A “substantial portion” is at least 10%, and can be at least 20%, at least 30%, at least 40%, at least 50%, at least 60%, at least 70%, at least 80%, or at least 90% or more.

It is contemplated that transit of protoporphyrin IX-Fe compounds across the BBB can be increased by co-administration of a compound which further disrupts the BBB. Examples of BBB disruptors include, but are not limited to, vasoactive compounds (e.g. bradykinin and RMP7) and molecules that cause osmotic disruption of the BBB (e.g. mannitol). High-intensity focused ultrasound (HIFU) can also disrupt the BBB (McDannold, Nathan; Vykhodtseva, Natalia; Hynynen, Kullervo (26 Oct. 2007), “Blood-Brain Barrier Disruption Induced by Focused Ultrasound and Circulating Preformed Microbubbles Appears to Be Characterized by the Mechanical Index”, Ultrasound in Medicine and Biology (Elsevier) 34 (5): 834-840, 21 January 2008). Convection-enhanced distribution can be used in bypassing the BBB. Other methods used to get through the BBB may entail the use of endogenous transport systems, including carrier-mediated transporters such as glucose and amino acid carriers; receptor-mediated transcytosis for insulin or transferrin; and the blocking of active efflux transporters such as p-glycoprotein.

Additionally, nanotechnology can be used to facilitate the transfer of drugs across the BBB (Silva, Ga. (December 2008). BMC Neuroscience 9: S4). Accordingly, in some embodiments, the protoporphyrin IX-Fe compounds intended for systemic delivery are attached to nanoparticles and/or are encapsulated in liposomes.

Transit of protoporphyrin IX-Fe compounds across the BBB can be increased by the route of administration. Injection of a protoporphyrin IX-Fe compound into the CSF (e.g. intracerebral injection) or intranasal administration are particular routes of administration contemplated for this purpose.

In some embodiments, a pharmaceutical composition comprises a protoporphyrin IX-Fe compound, and optionally a pharmaceutically acceptable carrier. The compositions encompassed by the technology described herein may further comprise at least one pharmaceutically acceptable excipient.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the protoporphyrin DC-Fe compound.

As described in detail below, the pharmaceutical compositions of the technology described herein comprising a protoporphyrin DC-Fe compound can be specially formulated for administration to a subject in solid, liquid or gel form. By way of non-limiting example, pharmaceutical compositions can be adapted for intravenous or intranasal administration. Additionally, a protoporphyrin IX-Fe compound can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Examples of dosage forms include, but are not limited to: solutions; aerosols (e.g., nasal sprays or inhalers); gels; liquids such as suspensions (e.g., aqueous or non-aqueous liquid suspensions, oil-in-water emulsions, or water-in-oil liquid emulsions), solutions, and elixirs; and sterile solids (e.g., crystalline or amorphous solids) that can be reconstituted to provide liquid dosage forms.

In some embodiments, parenteral dosage forms of a protoporphyrin IX-Fe compound can also be administered to a subject who has suffered CNS hemorrhage by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration to a patient, including, but not limited to, administration of DUROS®-type dosage forms, and dose-dumping.

Suitable vehicles that can be used to provide parenteral dosage forms of the protoporphyrin IX-Fe compound as disclosed within are well known to those skilled in the art. Examples include, without limitation: sterile water; water for injection USP; saline solution; glucose solution; aqueous vehicles such as but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Compounds that alter or modify the solubility of a pharmaceutically acceptable salt of a protoporphyrin IX-Fe compound as disclosed herein can also be incorporated into the parenteral dosage forms of the disclosure, including conventional and controlled-release parenteral dosage forms.

A composition comprising a protoporphyrin IX-Fe compound can be administered directly to the intranasal cavity of a subject in the form of an aerosol or by nebulization. For use as aerosols, a protoporphyrin IX-Fe compound in solution or suspension may be packaged in a pressurized aerosol container together with suitable propellants, for example, hydrocarbon propellants like propane, butane, or isobutane with conventional adjuvants. A protoporphyrin IX-Fe compound can also be administered in a non-pressurized form such as in a nebulizer or atomizer. In some embodiments, a protoporphyrin IX-Fe compound can also be administered directly to the airways or nasal mucosa in the form of a dry powder. For use as a dry powder, a protoporphyrin IX-Fe compound can also be administered by use of an inhaler. Exemplary inhalers include metered dose inhalers and dry powdered inhalers.

The protoporphyrin IX-Fe compounds can also be administered through mucosal routes. Mucosal dosage forms of the compositions comprising a modulator of a protoporphyrin IX-Fe compound as disclosed herein include, but are not limited to, sprays, aerosols, gels, solutions, emulsions, suspensions, or other forms known to one of skill in the art. See, e.g., Remington: The Science and Practice of Pharmacy, 21st Ed., Lippincott, Williams, and Wilkins, Philadelphia Pa. (2005); and Ansel's Pharmaceutical Dosage Forms and Drug Delivery Systems, 9^{th} Ed., Lippincott, Williams, and Wilkins, Philadelphia, Pa. (2011).

Examples of dosage forms and methods of administration that can be used to administer the active ingredient(s) described herein, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466,465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference in their entirety.

Suitable excipients (e.g., carriers and diluents) and other materials that can be used to provide mucosal dosage forms encompassed by this disclosure are well known to those skilled in the pharmaceutical arts, and depend on the particular tissue or organ to which a given pharmaceutical composition or dosage form will be applied. With that fact in mind, typical excipients include, but are not limited to water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with a protoporphyrin IX-Fe compound. For example, penetration enhancers can be used to assist in delivering the active ingredients to or across the tissue. Penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, dehydrates, co-crystals, solvates, polymorphs, anhydrous, or amorphous forms of the pharmaceutically acceptable salt of a protoporphyrin IX-Fe compound can be used to further adjust the properties of the resulting composition.

In some embodiments, a protoporphyrin IX-Fe compound can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm&Haas, Spring House, Pa. USA).

As disclosed herein, a protoporphyrin DC-Fe compound can be administered to a subject alone, or optionally in combination (e.g. simultaneously with, sequentially or separately) with one or more pharmaceutically active agents, e.g. a second therapeutic agent known to be beneficial in treating CNS hemorrhage, a condition in which CNS hemorrhage is known to be a complication or a condition which commonly occurs in patients who are also suffering a CNS hemorrhage. For example, exemplary pharmaceutically active compounds include, but are not limited to, those found in Harrison's Principles of Internal Medicine, 18^{th} Edition, Eds. A. Fauci et al. McGraw-Hill N.Y., NY; Physicians Desk Reference, 65^{th} Edition, 2011, Oradell N.J., Medical Economics Co.; Pharmacological Basis of Therapeutics, 12^{th} Edition, Brunton et al., 2010; United States Pharmacopeia, The National Formulary, USP XXXIV NF XIX, 2011; current edition of Goodman and Gilman's The Pharmacological Basis of Therapeutics; and current edition of The Merck Index, the complete contents of all of which are incorporated herein by reference. By way of non-limiting example, such therapeutic agents include antihypertensive agents, Factor VIIa, mannitol, acetaminophen, plasma, vitamin K, protamine, platelet transfusion, anticonvulsants, stress ulcer prophylactics, corticosteroids, and IV fluids.

Antihypertensive agents can include, but are not limited to metolazone, chlorthalidone, indapamide, bendroflumethiazide, chlorothiazide, hydrochlororthiazide, epitizide, torsemide, furosemide, ethacrynic acid, bumetanide, amiloride, triamterene, spironolactone, bucindolol, carvedilol, labetalol, tolazoline, terazosin, prazosin, phenoxybenzamine, indoramin, phentolamine, doxazosin, timolol, propranolol, pindolol, oxprenolol, nadolol, metoprolol, atenolol, guanfacine, clonidine, lercanidipine, isradipine, felodipine, amlodipine, nitrendipine, nimodipine, nifedipine, nicardipine, verapamil, diltiazem, aliskiren, aptopril, enalapril, benazepril, trandolapril, ramipril, quinapril, perindopril, lisinopril, fosinopril, valsartan, telmisartan, olmesartan, losartan, irbesartan, eprosartan, candesartan, Epelerenone, spironolactone, sodium nitroprusside, hydralazine, reserpine, guanethidine, moxonidine, methyldopa, and guanabenz. In particular, labetolol and nicardipine are often used to control blood pressure in patients suffering from CNS hemorrhage. Nimodipine is specifically used in subarachnoid hemorrhage patients to treat cerebral vasospasm.

Anticonvulsant agents can include, but are not limited to, fosphenyloin, carbamazepine, oxcarbazepine, acetazolamide, clonazepam, diazepam, divalproex sodium, ethosuximide, ethotoin, felbamate, gabapentin, lamotrigine, levetiracetam, mephenyloin, metharbital, methsuximide, methazolamide, oxcarbazepine, phenyloin, phensuximide, pregabalin, primidone, tiagabine, zonisamide, vigabatrin, valproic acid, trimethadione, and topiramate.

Stress ulcer prophylactics can include, but are not limited to, H2 antagonists and proton pump inhibitors. Examples of H2 antagonists include, but are not limited to, ranitidine and famotidine. Examples of proton pump inhibitors include, but are not limited to, omeprazole (brand names: Losec®, Prilosec®), lansoprazole (brandnames: Prevacid®, Zoton®), esomeprazole (brand names: Nexium®), pantoprazole (brandnames: Protonix®, Somac®), rabeprazole (brand names: Aciphex®, Pariet®), CS-526 (Sankyo), AZD0865 (Astra Zeneca) and soraprazan (Altana AG).

In some embodiments, a composition comprising a protoporphyrin IX-Fe compound and a pharmaceutically active agent can be administered to the subject in the same pharmaceutical composition or in different pharmaceutical compositions (at the same time or at different times). When administered at different times, a composition comprising a protoporphyrin IX-Fe compound and the additional pharmaceutically active agent can be administered within 5 minutes, 10 minutes, 20 minutes, 60 minutes, 2 hours, 3 hours, 4, hours, 8 hours, 12 hours, 24 hours of administration of the other. When a composition comprising a protoporphyrin IX-Fe compound and the pharmaceutically active agent are administered in different pharmaceutical compositions, routes of administration can be different. For example, a composition comprising a protoporphyrin IX-Fe compound can be administered by any appropriate route known in the art including, but not limited to intravenous and intranasal administration, and the pharmaceutically active agent is administered by a different route, e.g. orally, or a route commonly used in the art for administration of the pharmaceutically active agent.

In some embodiments, a composition comprising a protoporphyrin IX-Fe compound can precede, can be concurrent with and/or follow the pharmaceutically active agent by intervals ranging from minutes to weeks. In embodiments where a composition comprising a protoporphyrin IX-Fe compound and composition comprising a pharmaceutically active agent are applied separately to a cell, tissue or organism, one would generally ensure that a significant period of time did not expire between the time of each delivery, such that the composition comprising a protoporphyrin IX-Fe compound and a pharmaceutically active agent would still be able to exert an advantageously combined effect on the cell, tissue or organism.

In some embodiments, the technology described herein contemplates the use of a composition comprising a protoporphyrin IX-Fe compound and the practice of the methods described herein in conjunction with other therapies such as surgery, antihypertensive therapy, or supportive care.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

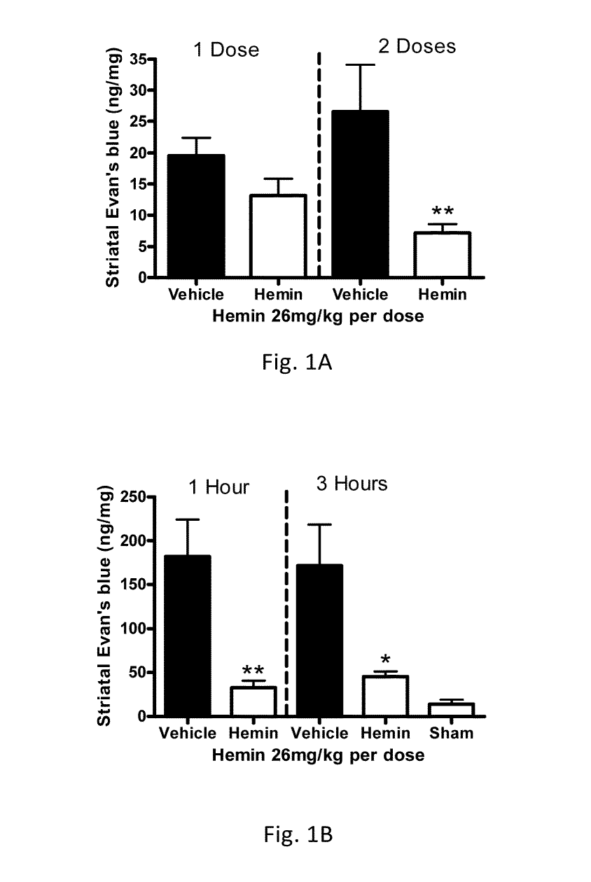

The effect of protoporphyrin IX-Fe compounds on subsequent exposure of nervous tissue to toxic doses of hemin was investigated. A mouse model of intracerebral hemorrhage (ICH) was used. ICH was induced in mice (6-7/condition, 9-10 weeks old) by injecting 25 μl autologous blood (FIG. 1A) into the right striatum. One hour after hemorrhage induction, mice were treated with (a) hemin at 26 mg/kg as a one-time dose, (b) hemin at 26 mg/kg at 1 hour after induction and then repeated at 25 hours after induction, or (c) sterile saline solution only. Intracerebral hemorrhage was also induced by striatal injection of collagenase. Mice were then treated with vehicle or hemin 1 hour later, with repeated dose 24 hours later. All treatments were administered via intraperitoneal injection. Three days after hemorrhage induction, brains were harvested after Evans blue injection, and slices were examined for Evan's blue fluorescence. Evan's blue fluoresces red when viewed with a DsRed filter. Reduced fluorescence was apparent in hemin-treated slices (data not shown). Evan's blue leakage into the striatal parenchyma was quantified as a marker of blood-brain barrier disruption (FIGS. 1A-1B).

Treatment with hemin attenuated blood-brain barrier disruption following simulated hemorrhage in a dose-dependent manner.

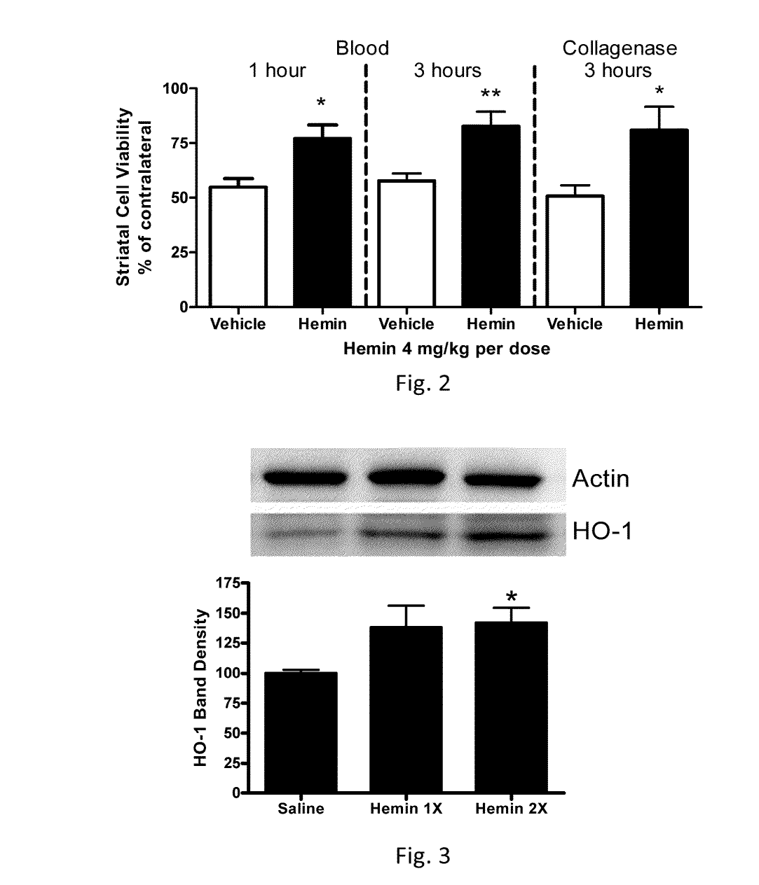

Whether protoporphyrin IX-Fe compounds could reduce hemorrhage-induced cell death in nervous tissues was investigated using the same mouse model of ICH. Intracerebral hemorrhage was modeled in 2-3 month old mice (5-6/condition) by striatal injection of autologous blood (25 μl) or collagenase (0.014 units), followed 1 or 3 hours later by 4 mg/kg hemin i.p. (repeated 24 h later) or vehicle. Five days after ICH, striatal cell viability was quantified by MTT assay (Chen et al., J Neurosurg 2011 114:1159-67) after striatal cell dissociation (FIG. 2). ICH was also modeled by striatal injection of 25 μA autologous blood, followed by hemin (26 mg/kg) or vehicle treatment at 3 and 27 hours after ICH. Increased MTT (3-(4,5-Dimethylthiazol-2-yl)-2,5-diphenyltetrazolium bromide) staining intensity in hemin-treated brain indicated increased tissue viability, compared with vehicle-treated brain (data not shown). In both experiments, treatment with hemin increased striatal cell viability following simulated CNS hemorrhage.

The response elicited by protoporphyrin IX-Fe compounds was investigated by administering 26 mg/kg hemin, or an equal volume of saline for 1 or 2 days via intraperitoneal injection. Expression of heme oxygenase-1 (HO-1) in striatal cells was assessed 24 hours after the last hemin dose. Four mice were used for each experimental group. Actin was used as a gel-loading control. HO-1 expression was quantified by immunoblotting as previously described (Neuropharmacology 2011 60:423-431); band density was analyzed using Kodak 1D software (Kodak, Rochester, N.Y.) (FIG. 3). Treatment with hemin increased expression of HO-1 in striatal cells.

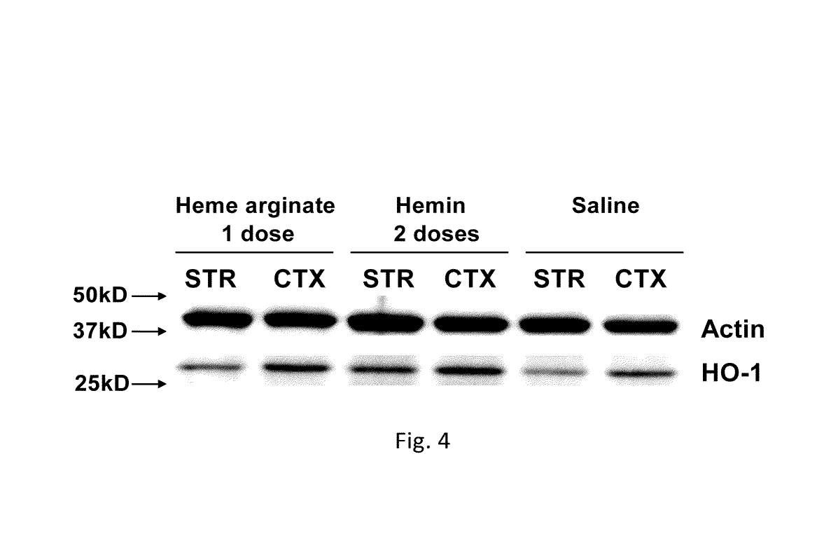

The effect of protoporphyrin IX-Fe compounds on HO-1 expression in nervous tissue was further investigated using heme arginate and examining the response in the cortex in addition to the striatum. Mice received 26 mg/kg hemin or heme arginate for 1 or 2 days, or an equal volume of saline. All treatments were administered intraperitoneally. HO-1 expression was quantified by immunoblotting as previously described (Neuropharmacology 2011 60:423-431). Treatment with both hemin and heme arginate injection increased HO-1 expression in the mouse striatum and cortex (FIG. 4).

What is claimed herein is:

1. A method of treating tissue damage resulting from a central nervous system (CNS) hemorrhage or a complication thereof in a subject having a CNS hemorrhage, the method comprising administering to the subject a protoporphyrin IX-Fe compound in a pharmaceutically acceptable carrier.

2. The method of claim 1, wherein the CNS hemorrhage is selected from the group consisting of:
an intracranial hemorrhage, a cerebral hemorrhage, an intracerebral hemorrhage, an intra-axial hemorrhage, an intraventricular hemorrhage, an intraparenchymal hemorrhage, an epidural hemorrhage, a subdural hemorrhage, and a subarachnoid hemorrhage.

3. The method of claim 1, wherein the complication is a stroke.

4. The method of claim 1, wherein the protoporphyrin IX-Fe compound is selected from the group consisting of:
hemin, hematin, hemoglobin, methemoglobin, heme arginate, and heme lysinate.

5. The method of claim 1, wherein the protoporphyrin IX-Fe compound is bound to albumin, haptoglobin or hemopexin.

6. The method of claim 1, wherein the protoporphyrin IX-Fe compound is conjugated or bound to a molecule which preferentially crosses the blood-brain barrier.

7. The method of claim 1, wherein the protoporphyrin IX-Fe compound is administered systemically.

8. The method of claim 1, wherein the protoporphyrin IX-Fe compound is administered intravenously.

9. The method of claim 1, wherein the protoporphyrin IX-Fe compound is administered intranasally.

10. The method of claim 1, wherein the protoporphyrin IX-Fe compound is administered locally to the site of the tissue damage.

11. The method of claim 1, wherein the method further comprises a step of diagnosing CNS hemorrhage in the subject prior to administering the protoporphyrin IX-Fe compound.

12. The method of claim 1, wherein the protoporphyrin IX-Fe compound is administered as one dose 1-24 hours after the subject has experienced a CNS hemorrhage.

13. The method of claim 1, wherein the protoporphyrin IX-Fe compound is administered as one dose 1-10 days after the subject has experienced a CNS hemorrhage.

14. The method of claim 1, wherein the protoporphyrin IX-Fe compound is administered as at least two doses with a time interval of 1-24 hours between the at least two doses.

15. The method of claim 1, wherein the protoporphyrin IX-Fe compound is administered as at least two doses with a time interval of 1-10 days between the at least two doses.

16. The method of claim 1, wherein the protoporphyrin IX-Fe compound is administered at a dose of 0.3 mg/kg to 100 mg/kg.

17. The method of claim 1, wherein the protoporphyrin IX-Fe compound is administered at a dose of 5 mg/kg to 75 mg/kg.

18. The method of claim 1, wherein the protoporphyrin IX-Fe compound is administered at a dose of 10 mg/kg to 50 mg/kg.

19. The method of claim 1, wherein the protoporphyrin IX-Fe compound is administered at a dose of 20 mg/kg to 30 mg/kg.

20. The method of claim 1, wherein the protoporphyrin IX-Fe compound is administered at a dose greater than 6 mg/kg/day.

21. A pharmaceutical composition comprising a protoporphyrin IX-Fe compound for the treatment of CNS hemorrhage in a subject.


SEQUENCE LISTING (8 sequences)

SEQ ID NO: 1 - protein, 609 aa, Homo sapiens [sequence omitted]

SEQ ID NO: 2 - DNA, 2264 bp, Homo sapiens
agtatattagtgctaatttccctccgtttgtcctagcttttctcttctgtcaaccccacacgcctttggcacaatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataacatcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaaaaaaaaaaaaaaaaaaa

SEQ ID NO: 3 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 4 - protein, 347 aa, Homo sapiens
MSALGAVIALLLWGQLFAVDSGNDVTDIADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 5 - DNA, 1461 bp, Homo sapiens
agatgccccacagcactgctcttccagaggcaagaccaaccaagatgagtgccctgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtatacaccttaaatgataagaagcagtggataaataaggctgttggagataaacttcctgaatgtgaagcagatgacggctgcccgaagccccccgagattgcacatggctatgtggagcactcggttcgctaccagtgtaagaactactacaaactgcgcacagaaggagatggagtgtacaccttaaacaatgagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccataaaaaaaaaaaaaaaaaaaaaaaaaaaaaa

SEQ ID NO: 6 - DNA, 1284 bp, Homo sapiens
agatgccccacagcactgctcttccagaggcaagaccaaccaagatgagtgccctgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtgtacaccttaaacaatgagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccataaaaaaaaaaaaaaaaaaaaaaaaaaaaaa

SEQ ID NO: 7 - protein, 462 aa, Homo sapiens
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 8 - DNA, 1635 bp, Homo sapiens
aactctatatagggagttcaactggtcacccagagctgtcctgtggcctctgcagctcagcatggctagggtactgggagcacccgttgcactggggttgtggagcctatgctggtctctggccattgccacccctcttcctccgactagtgcccatgggaatgttgctgaaggcgagaccaagccagacccagacgtgactgaacgctgctcagatggctggagctttgatgctaccaccctggatgacaatggaaccatgctgttttttaaaggggagtttgtgtggaagagtcacaaatgggaccgggagttaatctcagagagatggaagaatttccccagccctgtggatgctgcattccgtcaaggtcacaacagtgtctttctgatcaagggggacaaagtctgggtataccctcctgaaaagaaggagaaaggatacccaaagttgctccaagatgaatttcctggaatcccatccccactggatgcagctgtggaatgtcaccgtggagaatgtcaagctgaaggcgtcctcttcttccaaggtgaccgcgagtggttctgggacttggctacgggaaccatgaaggagcgttcctggccagctgttgggaactgctcctctgccctgagatggctgggccgctactactgcttccagggtaaccaattcctgcgcttcgaccctgtcaggggagaggtgcctcccaggtacccgcgggatgtccgagactacttcatgccctgccctggcagaggccatggacacaggaatgggactggccatgggaacagtacccaccatggccctgagtatatgcgctgtagcccacatctagtcttgtctgcactgacgtctgacaaccatggtgccacctatgccttcagtgggacccactactggcgtctggacaccagccgggatggctggcatagctggcccattgctcatcagtggccccagggtccttcagcagtggatgctgccttttcctgggaagaaaaactctatctggtccagggcacccaggtatatgtcttcctgacaaagggaggctataccctagtaagcggttatccgaagcggctggagaaggaagtcgggacccctcatgggattatcctggactctgtggatgcggcctttatctgccctgggtcttctcggctccatatcatggcaggacggcggctgtggtggctggacctgaagtcaggagcccaagccacgtggacagagcttccttggccccatgagaaggtagacggagccttgtgtatggaaaagtcccttggccctaactcatgttccgccaatggtcccggcttgtacctcatccatggtcccaatttgtactgctacagtgatgtggagaaactgaatgcagccaaggcccttccgcaaccccagaatgtgaccagtctcctgggctgcactcactgaggggccttctgacatgagtctggcctggccccacctcctagttcctcataataaagacagattgcttcttcgcttctcactgaggggccttctgacatgagtctggcctggccccacctccccagtttctcataataaagacagattgcttcttcacttgaatcaagggacctaaaaaaaaaaaa